(19) United States
(12) Patent Application Publication
(10) Pub. No.: US 2026/0054083 A1
(43) Pub. Date: Feb. 26, 2026

(54) MULTI-LOCUS TRANSCRANIAL MAGNETIC STIMULATION TRANSDUCER

(71) Applicant: AALTO UNIVERSITY FOUNDATION SR, Aalto (FI)

(72) Inventors: Jaakko NIEMINEN, Aalto (FI); Risto ILMONIEMI, Aalto (FI); Victor SOUZA, Aalto (FI); Juuso KORHONEN, Aalto (FI)

(73) Assignee: AALTO UNIVERSITY FOUNDATION SR, Aalto (FI)

(21) Appl. No.: 19/103,348

(22) PCT Filed: Aug. 12, 2022
(86) PCT No.: PCT/FI2022/050526

§ 371 Date: Feb. 12, 2025

(51) Int. Cl. A61N 2/00; A61N 2/02

(52) U.S. Cl. CPC A61N 2/006; A61N 2/02

(57) ABSTRACT

Disclosed is a multi-locus TMS (transcranial magnetic stimulation) transducer, including one or more transducer modules configured (i.e., adapted) to be positioned on a scalp of a subject for TMS, each of the one or more transducer modules including five or more coils for TMS of a cortical target of a subject.

### FIELD

The present invention relates to transcranial magnetic stimulation (TMS). In particular, the disclosure relates to multi-locus TMS.

### BACKGROUND

Transcranial magnetic stimulation allows one to induce a focused electric field (E-field) in the brain by applying a strong, rapidly changing magnetic field. TMS is performed routinely in thousands of hospitals worldwide for diagnostics (e.g., to measure corticospinal excitability), presurgical planning (to locate eloquent brain areas) and therapeutics (e.g., to treat depression or chronic pain). With present state-of-the-art equipment, a hand-held TMS coil is moved with the help of optically guided navigation so that cortical sites selected from individual magnetic resonance images (MRIs) are stimulated. Multi-locus stimulation with commercially available technology is cumbersome and at best limited to the stimulation of 2-3 distant cortical targets by using several bulky stimulators.

### OBJECTIVE

An objective is to alleviate the disadvantages mentioned above.

In particular, it is an objective to provide an improved multi-locus TMS transducer.

### SUMMARY

To overcome limitations, instrumentation and methods for multi-locus TMS (mTMS) have been developed, allowing one to stimulate cortical areas in a flexible manner. This may be done for neighboring cortical areas in sequence at 0.1-10000-millisecond time scales, significantly increasing the suitability of TMS for studying the interaction between cortical sites. The implementation of mTMS is made possible by advanced coil-design methods. In the context of the present invention, so-called minimum-energy (which may refer to requiring the least amount of energy) coils and coil sets (which may refer to transducers) may be produced to induce a desired E-field pattern in the cortex. These methods can be used for designing optimal coils and transducers that lie on planar or curved surfaces. As a result, the efficiency of TMS may be improved by a factor of two, for example. Planar coils may be used to provide an optimal setup but if the coils are allowed to span a large part of the scalp (which may be referred as having a large scalp foot-print) such wide coils can be impractical in mTMS. A key criterion for efficiency of a TMS coil or transducer is that it fits on the scalp. Any gap between the coil and the scalp may drastically increase the required energy supplied by the stimulator electronics, making it impossible to achieve the desired E-field strength in the cortex if the gap is too large.

According to a first aspect, a multi-locus TMS transducer comprises one or more transducer modules adapted to be positioned on a scalp of a subject for TMS. This allows the transducer module(s) to follow the contours of the scalp, thereby also allowing minimizing the gap between the transducer modules and the scalp. With multiple transducer modules, the modular configuration allows efficient multi-locus TMS, where the stimulating E-field can be controlled electronically without coil movement by adjusting currents in the coils of the transducer modules. Importantly, each of the one or more transducer modules comprises five or more coils for TMS of a cortical target of the subject. This allows moving any maximum of the induced E-field in two dimensions across the scalp and rotating the E-field maximum about an axis, which may be substantially perpendicular to an inner skull surface. In particular, this allows controlling the position and orientation of the global maximum of the E-field, up to fine control. This also allows controlling the position of the center of gravity of any region of the E-field, e.g., within the region where the E-field amplitude exceeds a threshold value (such as 90 or 95% of the global maximum of the E-field), and/or the associated orientation of the E-field. The threshold value may be relative (as in the example above) or ab-solute. This kind of averaging across the region where the E-field is of high amplitude may be useful to avoid problems with the global maximum. Due to the complex conductivity geometry of the cortical region below the scalp, the global E-field maximum may exhibit jumping from gyrus to gyrus even when the overall E-field profile changes more smoothly.

It is noted that one could actually think that six coils would be needed to achieve this behavior. With the electric field having two components, the first component may be called, for example, an “x component” and the second component a “y component”. To control the x component of the E-field, one could expect to need three coils for the three degrees of freedom: strength and 2D position. Similarly, one could expect to need three coils to control the three degrees of freedom for the y component of the E-field: its maximum and 2D position. However, it is found that the physics of the problem guarantees that only five degrees of freedom are needed for rotation and small movements. This is applicable for TMS. With only five coils, the maximum may be controllable only within a limited region. More coils (a few tens of coils, for example, depending on the required level of the focality of the E-field maximum) may be utilized to be able to control the E-field maximum substantially across the whole cortical surface. The location of the maximum is not a linear metric.

Accordingly, the transducer, and the one or more transducer modules, may be configured for the magnitude and the two positions of the E-field maximum to be controlled for both of the components of the E-field by the five or more coils. For any or each of the transducer modules this can be done within a limited region. With multiple transducer modules this can be done across an extended area, beyond the reach of a single transducer module.

It should be noted that the transducer, and the transducer module(s), may be configured for the cortical target to be changed. This may be done by varying how electric currents are driven through the five or more coils. The transducer may be configured for two distinct targets to be stimulated in succession with the same of the five or more coils, i.e. utilizing the same transducer module. This may be done in rapid succession, for example 0.5-100 ms apart.

In an embodiment, the windings of any or all of the five or more coils span a three-dimensional volume. This way, the coil(s) are not packed against the scalp but can be arranged to protrude away from the scalp, thereby providing extended space for coil design. This allows improved winding efficiency for the coil(s), which in turn allows improved energy efficiency for the transducer as a whole. In a further embodiment, the windings of any or all of the one or more transducer modules at the bottom of the corresponding transducer module (i.e. at the end of the transducer module configured for positioning against the scalp) are transversely confined within a bottom region and spread out transversely beyond the bottom region when longitudinally away from the bottom. This allows providing extended space for coil design not only in the depth dimension (i.e. longitudinal dimension) but also in one or both of the lateral dimensions (i.e. those in parallel with the surface of the scalp). The lateral extent of the coils may thus be made larger than the scalp footprint of the corresponding transducer module(s) allowing flexibility in coil design.

In an embodiment, the bottom of any or all of the one or more transducer modules is curved for following the scalp. This allows the gap between the transducer module(s), or the coils thereof, and the scalp to be reduced, thereby improving efficiency of the transducer module(s) for TMS by reducing the required energy supplied by the stimulator electronics.

In an embodiment, the bottoms of any or all of the one or more transducer modules are shaped as hexagons and/or pentagons. This allows multiple transducer modules to be positioned close to each other so that the gaps between the transducer modules are minimized, thereby allowing improved presence of the E-field also at the boundaries of the transducer modules.

In an embodiment, the one or more transducer modules comprise a plurality of transducer modules adapted to be positioned adjacent to each other on the scalp of the subject for TMS. The modular configuration of the transducer allows efficient multi-locus TMS, where a cortical target may be stimulated even beyond the effective stimulation regions of any individual transducer modules.

In an embodiment, the bottoms of the plurality of transducer modules form one or more patterns of a pentagon surrounded with one or more hexagons and/or one or more pentagons, in particular with five such hexagons, which may encircle the pentagon. This allows a close-fitting configuration of the transducer modules.

In an embodiment, the bottoms of the plurality of transducer modules form one or more patterns of a hexagon surrounded with three hexagons and three pentagons. The surrounding hexagons and pentagons may alternatingly encircle the (central) hexagon. This allows another close-fitting configuration of the transducer modules.

In an embodiment, the bottoms of the plurality of transducer modules form one or more patterns of a central transducer module encircled by a plurality of encircling transducer modules, wherein the bottom for any or all of the encircling transducer modules has an irregular shape for extending the scalp footprint of the corresponding transducer module away from the central transducer module. This allows the scalp footprint of the corresponding encircling transducer module(s) to be made much larger than that of the central module. This, in turn, allows spanning the coil windings in the corresponding encircling module(s) within a larger space, thereby allowing increase in stimulation efficiency.

In an embodiment, the transducer is configured for simultaneously activating any or all of the five or more coils of two or more neighboring transducer modules of the plurality of transducer modules for stimulating a single cortical target. This allows stimulating a cortical target beyond the reach of an individual transducer module, for example at a boundary region between the two or more transducer modules.

In an embodiment, the five or more coils are adapted for generating a signal space of five or more dimensions with each of the five or more coils corresponding to an orthogonal basis vector of the signal space. This allows an improved coil design, where electric currents for the five or more coils do not couple to each other. For this purpose, the five or more coils may have negligible mutual inductance.

In an embodiment, the five or more coils for any or all of the plurality of transducer modules have a coupling coefficient for mutual inductance less than 0.1. In a further embodiment, the coupling coefficient is less than 0.05. It is noted that the mutual inductance M of two coils may be calculated as M=k*sqrt(L1*L2), where L1 and L2 are the self-inductances of the two coils, respectively, and k is the coupling coefficient for mutual inductance. Here, “sqrt” indicates a square root of the product within the following parentheses. Having low mutual inductances, as indicated in the embodiments, between the coils in a transducer is beneficial, as that reduces the voltages the coils induce in each other during TMS pulses. In an embodiment, the five or more coils comprises one or more round coils.

In an embodiment, the five or more coils comprises one or more figure-of-eight coils. In a particular embodiment, this includes two figure-of-eight coils, which may be perpendicular with respect to each other. Herein, a “figure-of-eight coil” may refer to a coil forming two loops or two sets of loops with its windings crossing in between, thereby forming a shape reminiscent of the figure “8”. The two figure-of-eight coils may be rotated with respect to each other about their longitudinal axis, which may be defined by the common point of crossing of the two coils. When perpendicular, the rotation may be substantially 90 degrees.

In general, any coils disclosed herein may form loops or sets of loops. The coils, including round coils and figure-of-eight coils, may have tightly wound loops (multiple turns next to each other, touching or nearly touching throughout the whole winding path). This can then form a set of loops for the coil, where the loops in the set may be stacked to follow each other. To increase the efficiency, the coil windings may be spanned, for example as shown in FIG. 1, so that the windings of a loop of a coil, such as a figure-of-eight coil, touch the windings of other loops near the common point. When moving away from the common point, a gap may grow between the loops.

In an embodiment, the five or more coils comprises one or more four-leaf clover coils. In a particular embodiment, this includes two four-leaf clover coils, which may be rotated about a common longitudinal axis. The rotation may be about 45 degrees, for example 40-50 degrees, so that a substantially eight-leaved pattern may be formed by the two coils. Herein, a “four-leaf clover coil” may refer to a coil forming four loops or four sets of loops with its windings crossing in a single region or a point in between, thereby forming a shape reminiscent of a four-leaf clover.

In an embodiment, the five or more coils comprises one round coil, two figure-of-eight coils, which may be perpendicular with respect to each other, and two four-leaf clover coils, which may be rotated about a common longitudinal axis with respect to each other, for example as described above.

In an embodiment, the five or more coils have the same axis of symmetry. This may be the longitudinal axis, i.e. the axis substantially perpendicular to the scalp when the transducer is positioned on the scalp for TMS.

The present solution allows mTMS to be provided in a controllable manner. In particular, it allows more or less continuous mTMS across the scalp, or any regions of interest thereof.

It is to be understood that the aspects and embodiments described above may be used in any combination with each other. Several of the aspects and embodiments may be combined together to form a further embodiment of the invention.

### BRIEF DESCRIPTION OF THE DRAWINGS

The accompanying drawings, which are included to provide a further understanding and constitute a part of this specification, illustrate examples and together with the description help to explain the principles of the disclosure. In the drawings:

FIG. 1 illustrates TMS with a transducer according to an example,

FIG. 2 illustrates a transducer according to two examples in a coronal view and a top view,

FIG. 3a illustrates shapes for transducer modules according to examples,

FIG. 3b illustrates a pattern for transducer modules according to an example,

FIGS. 4a and 4b illustrates examples of a transducer module,

FIG. 5 illustrates a further example of a transducer module, and

FIG. 6 illustrates further shapes for transducer modules according to examples.

Like references are used to designate equivalent or at least functionally equivalent parts in the accompanying drawings.

### DETAILED DESCRIPTION

The detailed description provided below in connection with the appended drawings is intended as a description of examples and is not intended to represent the only forms in which the example may be constructed or utilized. However, the same or equivalent functions and structures may be accomplished by different examples.

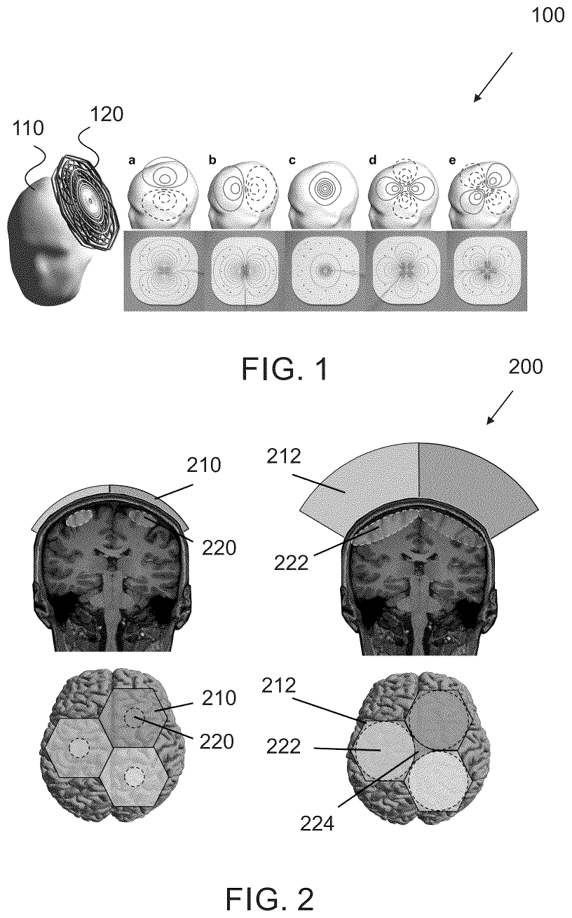

FIG. 1 shows an example 100 of TMS on a subject. The subject has a scalp 110 against which a TMS transducer 120 is positioned for TMS. In this example, the transducer is a planar transducer and it allows controlling the stimulation location, direction, and intensity in a cortical area of about 30 mm×30 mm providing electronically movable focal targeting in that area. The transducer may have five separate coils for TMS. In sub-figures a-e, an example of coil windings for each of the five coils is illustrated.

A multi-locus TMS (mTMS) transducer (also “the transducer”) may be provided with a wider coverage of the cortex of the subject. The transducer may comprise one or more transducer modules, in particular a plurality of transducer modules, each of which may be adapted to cover a different portion of the scalp of the subject. As the transducer is modular, it may comprise or consist of separate units, each of which may be adapted to be placed directly on the scalp, separately or together with any or all of the other transducer modules. The transducer modules may be adapted to be positioned adjacent to each other on a scalp of a subject for TMS. This may be done with or, in particular, without a gap between the transducer modules. The transducer may comprise a frame supporting any or all of the transducer modules and/or binding them together. Any or all of the transducer modules may be enclosed in a casing, which may be part of the transducer. The casing may be common to said transducer modules. The frame may be part of the casing. The casing and/or the frame may have flexibility allowing it to be deformed allowing it to follow the shape of the scalp.

Each of the one or more transducer modules may comprise five or more coils for TMS of a cortical target of the subject. The coils can be configured for providing an E-field at the cortex for TMS. Having five coils for a module allows controlling the E-field both translationally across the cortex and rotationally about a rotation dimension, which may be perpendicular or substantially perpendicular with respect to the cortical region for TMS and/or an inner skull surface at the location of an E-field maximum/cortical target. The transducer may be configured for electric current to be provided to the coils for moving an electric field maximum corresponding to a cortical target (e.g. in two dimensions across the cortex). It may also be configured for electric current to be provided to the coils for rotating an electric field maximum corresponding to a cortical target. The five or more coils may have a negligible mutual inductance with each other. In general, the five or more coils may be adapted for generating a signal space of five or more dimensions with each of the five or more coils corresponding to an orthogonal basis vector of the signal space.

As a contrasting example, one may picture having only three-coil modules, for example with simple round coils along three orthogonal axes. Two of those coils may touch the scalp with one of their edges and can thus be similar to figure-of-eight coils (they produce an electric field maximum below the edge touching the scalp, the E-field maximum being directed dominantly along the winding direction; if one starts with a figure-of-eight coil and bends it windings upwards, one gets this type of a round coil). Those two coils would thus allow rotating the E-field maximum. The third round coil may then, in principle, function similarly to any round coils included in the present invention. With such three-coil modules one is thus lacking the four-leaf clover coils. In such three-coil modules, the coils would be relatively small and thus inefficient (the magnetic fields they generate and the corresponding electric field decay fast as a function of distance). A figure-of-eight coil (that follows the scalp) is more efficient in inducing an electric field than a round coil that is touching the scalp with its edge.

The effect of more than three coils can also be explained by first understanding that the vector-valued electric field consists substantially of two perpendicular components, _{[JN1]}both of which can be nearly tangential to the inner skull surface (for non-spherical geometries, such as a head, there can be three components, but one of them is small compared to the others). As indicated above, the present solution allows controlling the strength and location (in two dimensions across the scalp) of the maximum of both of these components of the E-field (six parameters in total). As indicated above, the physics of the problem then couples the two components in a way that reduces the number of needed degrees of freedom to five.

The transducer can be made flexible allowing it to fit various head shapes and sizes to minimize the coil-cortex distance. For this purpose, the individual transducer module(s) may be flexible, e.g. with the five or more coils being flexible. The five or more coils may also be rigid, however, in which case the modular structure of the transducer allows all of the separate transducer modules to be placed directly on the scalp.

FIG. 2 shows two examples of the transducer 200. On the left, the one or more transducer modules 120 are two-dimensional, i.e. the coils of the transducer modules are arranged to form a two dimensional surface for positioning against the scalp. The surface may be planar or curved, the latter option allowing improved match against the scalp. Within a transducer module, the coils may be stacked on top of each other. The two-dimensional form results from the individual coils being arranged to form a two-dimensional surface for TMS.

On the right, the one or more transducer modules 210 are three-dimensional, i.e. the coils of the transducer modules, in particular the windings thereof, are arranged to span three dimensions. Within a transducer module, the coils may be arranged to be intertwined. The three-dimensional form results from the individual coils being arranged to have their windings extend in all three dimensions for TMS. This may hold for any or all of the five or more coils. In particular, one may have at least five coils with windings for TMS extending in all three dimensions. The five or more coils may be positioned in a nested manner with respect to each other.

The three-dimensional form allows improved efficiency in stimulating the areas under the module borders, in particular. This is illustrated in FIG. 2 (on top: coronal view; at bottom: top view), where the coil windings of a transducer module 120, 210 may extend up to the borders of the transducer module. In 2D, an illustrated transducer module 120 consists of planar coils stacked on top of each other, whereas in 3D, the coils of the transducer module 210 span a volume. In 2D, the coils of a single transducer module can be used to move the stimulated spot within a region 220 that is small in comparison to the scalp footprint of the module. In 3D, the coils of a single transducer module can be used to move the stimulated spot within a region 222 that is comparable to the scalp footprint of the module: more than half the scalp footprint or even substantially equal to the scalp footprint. On the other hand, the transducer may also be configured for the movement of the stimulated spot to be limited within a region smaller than the scalp footprint, for example to 10-50 percent of the scalp footprint or smaller. Even if the transducer, or transducer modules, configured in this manner may not allow having a large continuous region within which the spot can be moved, they can still allow controlling the stimulation within relatively large regions around the multiple module centers. This would still be an improvement over conventional transducers having planar transducer modules. The transducer may be configured for the coils of any or all neighboring transducer modules to be simultaneously activated. This can be done to allow the cortical area available for stimulation to be further extended. This allows extending the cortical area for stimulation beyond that of the corresponding area for any single transducer module, into a boundary region 224 between two or more transducer modules. For clarity, in the examples illustrated in FIG. 2 only two (coronal view) and three (top view) transducer modules are shown. In the simplest configuration, the one or more transducer modules consist of a single transducer module. While it is a minimum for the plurality of transducer modules to consist of two transducer modules, it should be understood that the number of these transducer modules may typically be larger than two or three, for example ten or more.

Transducer modules, or the coils thereof, with a three-dimensional winding geometry allow a three-dimensional return path for the current injected therein for TMS. In two dimensions, the current loops at the borders typically need to be squeezed (which may result in inefficient TMS) to form complete loops. In three dimensions, no squeezing is needed, as the current loops for the transducer module, or the coils thereof, may be completed by return wires above the other wires. This allows improved design efficiency.

By allowing the coil windings to span a three-dimensional volume, the efficiency of the coils can be improved from planar designs having a corresponding scalp footprint. The three-dimensional transducer module(s) allow maximizing the area over which the stimulated spot on the cortex can be moved, given a fixed scalp footprint of the transducer. With three-dimensional transducer modules, the transducer will be able to stimulate a sufficiently large area of the cortex. This overcomes a fundamental limitation of existing technology, where 2D transducers are able to adjust the stimulation target only within a small cortical region. For example, even an optimized planar 5-coil mTMS transducer, for example as illustrated in FIG. 1, is relatively large although it can adjust the stimulation only within a ˜10-cm2 cortical region. With a three-dimensional winding geometry, the (three-dimensional) transducer as disclosed herein can be provided with a significantly smaller size.

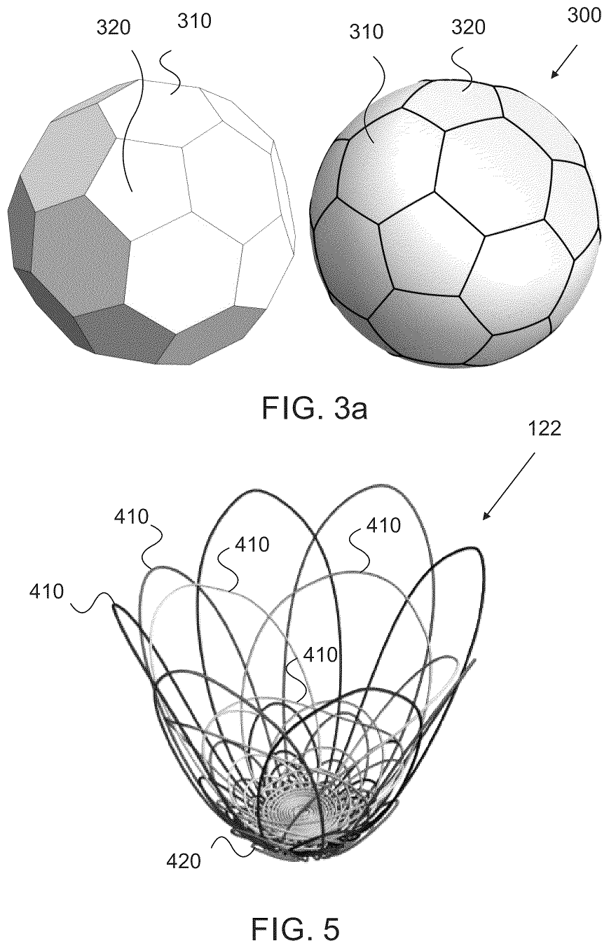

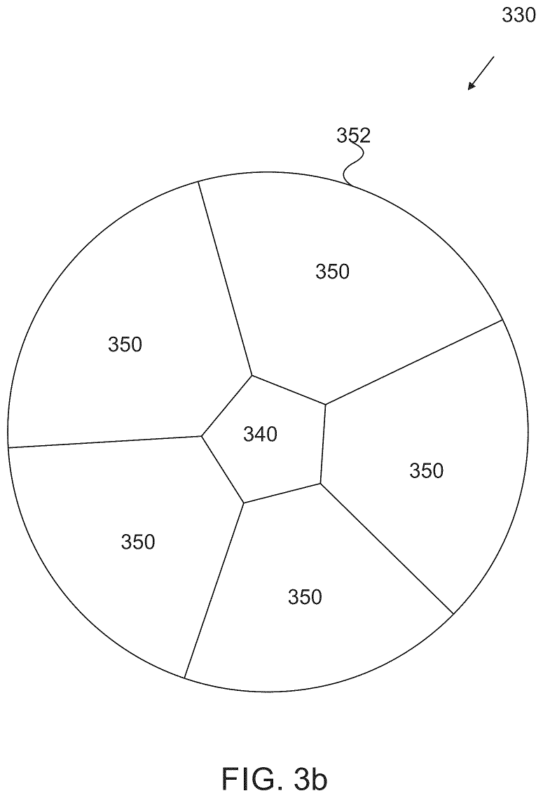

FIG. 3a illustrates some possible shapes for transducer modules. The one more of transducer modules may comprise or consist of transducer modules having a polygonal shape. This may include hexagonal 310 (as in the examples illustrated in FIG. 2) and/or pentagonal 320 shapes. The shape may correspond the borders of the transducer module, in particular at the end of the module defining its scalp footprint. The borders can thus be taken to correspond to the shape of the bottom of the transducer module. This may directly correspond to the shape of any or all of the five or more coils at the bottom of the module. The bottom may be planar or curved, the latter allowing the module, or the coils thereof in particular, to follow the shape of the scalp, thereby reducing or minimizing the distance between the coil(s) and the cortex. On the left in FIG. 3a, the modules have planar bottoms. On the right in FIG. 3a, the modules have curved bottoms. The number, positioning and shape of the modules may be such that they efficiently cover any relevant cortical regions. The illustrations are provided in terms of spheres 300 but in practice the transducer may have the modules arranged in an open pattern, such as an opened spherical pattern. It may comprise, at least, a semi-spherical pattern of transducer modules but smaller arrangement is also possible. The spherical or semi-spherical pattern need not correspond to an exact (semi-)sphere but it may be adapted for the shape of a scalp. The transducer may comprise a pentagon-shaped transducer module surrounded with hexagons, for example with five hexagons each joining a different side of the pentagon. Alternatively or additionally, it may comprise a hexagon-shaped transducer module surrounded with hexagons and pentagons, for example with three pentagons and three hexagons, each joining a different side of the hexagon at the center. Each surrounding/encircling hexagon may also join two surrounding/encircling pentagons and vice versa.

FIG. 3b illustrates an example of a pattern for the plurality of transducer modules, i.e. when the one or more transducer modules comprises two or more transducer modules. The plurality of transducer modules may be arranged so that they form one or more patterns 330 of a central module 340 (of the plurality of transducer modules) surrounded by multiple modules 350 (of the plurality of transducer modules), e.g. five or six, encircling the central module. The central module may have any suitable shape, such as a pentagon or hexagon. It may have a flat or a curved bottom. In general, it may be in accordance with any example described herein for the plurality of transducer modules. The encircling modules 350 may then form a ring of modules around the central module. The encircling modules may have shapes, e.g. polygonal shapes, that fit against each other and/or the central module, thereby leaving no gaps between any pair of neighboring encircling modules and/or between the central module and the encircling modules. The outer border 352 of any or all of the encircling modules may have any shape, e.g. a straight or a curved one, so that the overall shape of the pattern is not limited to that of a regular polygon. In particular, the outer border for any or all of the encircling transducer modules may have such a shape that the encircling module in question, as a whole, has an irregular shape, for example so that against the central module it has a straight border but away from the central module it has a curved border. Alternatively, the outer border away from the central module may be straight or piece-wise straight but the bottom as a whole may be irregular. As a result, the pattern 330, as a whole, may have any shape, for example a partially or fully rounded shape, or a circular or oval shape in particular. Having any all of the encircling modules with an irregular outer boundary allows extending the scalp footprint of the corresponding transducer module(s). This, in turn, allows spanning the coil windings in the corresponding encircling module(s) within a larger space, thereby allowing increase in stimulation efficiency. In this configuration, the encircling modules could thus be larger than the central module, even much larger.

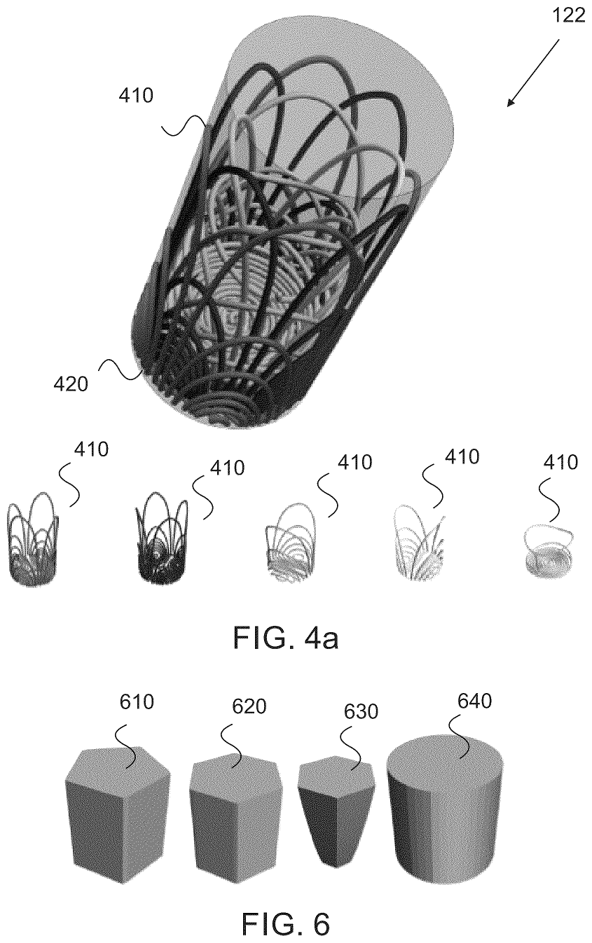

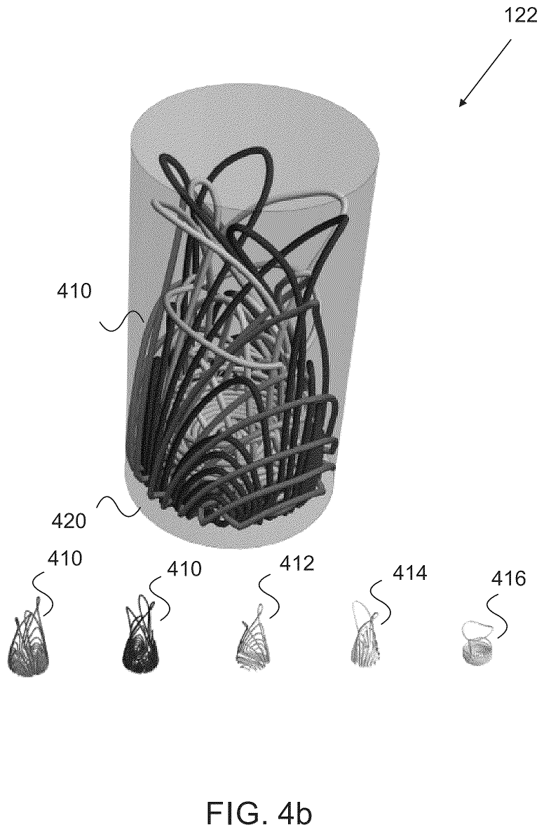

On the other hand, the plurality of transducer modules may also consist of only two such modules. This may be highly useful in some cases due to its simplicity. The two modules in such a case may be arranged to connect to each other along a long edge on the scalp. One of the two modules may be arranged to be placed against the left side of the scalp of the subject and the other against the right side of the scalp of the subject. In general, the number of the transducer modules in the plurality of transducer modules may be anything from two, three or four to tens of modules. In a particular embodiment, the plurality of transducer modules comprises at least five or six transducer modules. FIGS. 4a and 4b show examples of a transducer module 210. The transducer modules are three-dimensional as they comprise coils 410 spanning three dimensions and protruding away from the bottom 420 of the module, the bottom defining the scalp footprint of the module. In both examples, the transducer module comprises five coils, which correspond to the five (or more) coils for TMS. For clarity, each individual coil is separately illustrated below the module. In a first example, the windings of any of the coils does not cross the windings of another coil. This is illustrated in FIG. 4a. In a second example, the windings of some or all of the coils go through the winding loops of another coil. This is illustrated in FIG. 4b, where the windings of the third coil 412 and the fourth coil 414, in particular, go through the winding loops of the fifth coil 416. In any case, the bottom of the coils may have the same shape for each of the five coils, as is the case in the illustrated examples, where the bottom is circular. The bottom of any or all of the coils may have the same shape and/or size as the bottom of the module, as is the case in the illustrated examples as well. In the illustrated examples, the windings of the coils are confined within a bottom of the transducer module, i.e. they do not spread (laterally) beyond the bottom even when protruding away from the bottom (in the longitudinal dimension).

FIG. 5 shows an example of a transducer module 210, which can otherwise be in accordance with any of the examples presented above but now has windings of any or all of the five or more coils 410 spreading out (laterally) beyond the bottom 420 away (longitudinally) from the bottom. The windings at an upper part of the module thus covers a wider space than the windings at the bottom of the module. Or, in another way, the windings of any or all of the five or more coils extend laterally beyond a bottom region of the transducer module. The bottom region may correspond to the scalp footprint of the transducer module. Or in other words, it may correspond to the physical transverse extent of the transducer module at the bottom of the transducer module. The windings may spread out in this manner only along a part of the circumference or along the full circumference of the scalp footprint. The windings for any or all of the coils may spread out symmetrically, for example in a conical pattern. A transducer module with spread out coil windings is also in accordance with the examples of FIG. 2 (right side). As illustrated therein, the windings of neighboring modules may spread out so that the neighboring transducer modules abut against each other also away from the bottom of said transducer modules, for example all the way to the top of the transducer modules.

FIG. 6 shows examples of different shapes of a three-dimensional transducer module. This includes a transducer module with a pentagonal shape 610, a hexagonal shape 620, a tapered shape 630 and a circular shape 640, but various other alternatives are possible as well. In each case, the shape may correspond to the shape of the bottom and/or top of the transducer module. In particular, it may correspond the shape of the windings of any or all of the five or more coils at the bottom the transducer module. Using pentagonal and/or hexagonal shapes for the coil(s) at the bottom allows minimizing gaps between neighboring transducer modules. As illustrated, the size of the transducer module at the top may be equal or larger than the size of the transducer module at the bottom. The shapes for the plurality of transducer modules may be the same or different for any or all of the transducer modules.

Energy-optimized 3D TMS coils and 3D mTMS transducers may be designed, for example, by expressing the current density corresponding to a coil on a divergence free basis. This can be done in a mesh such as a polygonal mesh and/or a polyhedral mesh. An example is a tetrahedral mesh or a triangular 3D mesh surface. One example of such a basis is provided by the efficient divergence-free 3D RWG (Rao-Wilton-Glisson) basis functions. To extract the discrete coil winding paths, one may apply, for example, dual (3D) stream functions. These have previously been used to model and visualize flows in 3D.

An example of a coil design algorithm is as follows. First, volume V_{transducer} may be specified within which the coil windings will reside. This may correspond to the volume of the transducer, as defined by its outer boundary. The volume may be simply connected or not simply connected and may consist of one or more regions. For computational purposes, the coil volume may be discretized, for example into tetrahedral or hexahedral elements. Next, a head model (or at least its geometry and electrical conductivity) and the set of locations within the head model where the electric field is to be computed may be specified. The model may be, for example, a realistic head model, which may be based, for example, on magnetic resonance imaging and/or computed tomography data of one or more individuals, or a simplified head model, such as a spherical head model. The electric field may be computed, for example, on a set of points on the cortex. Alternatively or additionally, the electric field may be computed on a set of points on the scalp and/or on the face or other regions of interest.

To follow, the set of intended electric field patterns in the head model (i.e., the set of electric field patterns the coil set should be able to produce) may be specified. The number of electric fields in this set may be referred to as N_{E-field}. The specifications may include any or all of the following: the intended location, intensity, and direction of the maximum of each electric field. Additional specifications may be given to constrain the shape of some or of all the electric fields in more detail, for example by requiring that the electric field intensity at certain locations will be below a threshold value. Alternatively, a full description (orientation, intensity) of each electric field may be given on each location.

A set of current density distributions {Jᵢ(r)|i=1, . . . , N_{coil}} in the discretized volume V_{transducer} may be determined corresponding to the coil windings. For this purpose, various approach may be used. In a first approach the current density Jᵢ(r) (i=1, . . . , N_{E-field}) may be optimized within the coil volume that induces the i:th intended electric field. This may be repeated for all N_{E-field} intended electric fields. The set of Jᵢ(r) (i=1, . . . , N_{E-field}) may then be processed to generate a set of J′ᵢ(r), i=1, . . . , N_{coil}, the (linear) combination of which can be used for mTMS. The N_{coil} current densities may correspond, for example, to the first N_{coil} principal components of the set Jᵢ(r) (i=1, . . . , N_{E-field}). In a second approach, the set of Eᵢ(r) (i=1, . . . , N_{E-field}) may be processed to generate a set of E′ᵢ(r) (i=1, . . . , N_{coil}), the (linear) combination of which reproduces the original set of N_{E-field} electric fields to a threshold. The N_{coil} electric fields may correspond, for example, to the N_{coil} first principal components of the set N_{E-field}. Then, optimization may be performed for the current densities J′ᵢ(r), i=1, . . . , N_{coil} that correspond to the E′ᵢ(r).

The set of J′ᵢ(r), i=1, . . . , N_{coil} may be discretized to obtain the winding paths for the coil windings. In particular, the discretization may be done so that the set of coil windings closely approximate the set of current densities.

To find the current density corresponding to a given electric field, a set of divergence-free basis functions for the current density in the discretized V_{transducer} may be defined. As indicated above, one example of such a basis is provided by the efficient divergence-free 3D RWG basis functions. The weights of the basis functions may be found out, for example, by solving a convex optimization problem to minimize the total magnetic field energy associated with the current density with the constraints of the optimization problem specifying the desired properties of the electric field. Such convex optimization problems can be solved efficiently, for example, with the interior point method.

The electric field computation may be implemented, for example, by utilizing any or all of the following: a boundary element method, a finite element method and analytical formulas.

The optimization may be carried out in various different ways. With a single head model and a single coil volume, one may repeat the optimization with different relative placements of the head and the coil volume to obtain “average” coil windings that allow efficient stimulation over multiple cortical regions instead of being optimal for a single cortical region only. With a single coil volume, one may further repeat the optimization with different head models to obtain coil windings that generalize well over different heads. One may utilize symmetries in the optimization to limit the number of independent basis functions to ultimately obtain coil modules that contain coils with some symmetry features. In case the coil volume consists of multiple identical but distinct regions, one may require that the final coil sets within similar subregions are similar to each other to reduce the number of different coil modules that need to be manufactured.

As an example, “Coil optimisation for transcranial magnetic stimulation in realistic head geometry” (http://hdl.handle.net/10138/297793, open access, ISSN 1935-861X) describes a method for optimizing a single TMS coil on a 2D surface (triangular mesh). Also, “Multi-locus transcranial magnetic stimulation-theory and implementation” (https://doi.org/10.1016/j.brs.2018.03.014, open access) describes a method for optimizing a transducer (coil set) on a 2D surface (triangular mesh). The approach presented in either of the papers can be adapted for the needs of 3D coil optimization, for example for providing the five or more coils for the transducer in accordance with the present disclosure, by replacing the 2D basis functions with 3D basis functions.

The different functions discussed herein may be performed in a different order and/or concurrently with each other.

Any range or device value given herein may be extended or altered without losing the effect sought, unless indicated otherwise. Also any example may be combined with another example unless explicitly disallowed.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item may refer to one or more of those items.

The term ‘comprising’ is used herein to mean including the method, blocks or elements identified, but that such blocks or elements do not comprise an exclusive list and a method or apparatus may contain additional blocks or elements.

Numerical descriptors such as ‘first’, ‘second’, and the like are used in this text simply as a way of differentiating between parts that otherwise have similar names. The numerical descriptors are not to be construed as indicating any particular order, such as an order of preference, manufacture, or occurrence in any particular structure.

Expressions such as ‘plurality’ are in this text to indicate that the entities referred thereby are in plural, i.e. the number of the entities is two or more.

Although the invention has been described in conjunction with a certain type of apparatus and/or method, it should be understood that the invention is not limited to any certain type of apparatus and/or method. While the present inventions have been described in connection with a number of examples, embodiments and implementations, the present inventions are not so limited, but rather cover various modifications, and equivalent arrangements, which fall within the purview of the claims. Although various examples have been described above with a certain degree of particularity, or with reference to one or more individual embodiments, those skilled in the art could make numerous alterations to the disclosed examples without departing from the scope of this specification.

1. A multi-locus TMS (transcranial magnetic stimulation) transducer, comprising:
one or more transducer modules configured to be positioned on a scalp of a subject for TMS, each of the one or more transducer modules including five or more coils for TMS of a cortical target of a subject.

2. The transducer according to claim 1, wherein windings of any or all of the five or more coils span a three-dimensional volume.

3. The transducer according to claim 2, wherein the windings of any or all of the one or more transducer modules at a bottom of a corresponding transducer module are transversely confined within a bottom region and spread out transversely beyond the bottom region when longitudinally away from the bottom.

4. The transducer according to claim 3, wherein the bottom of the one or more transducer modules is curved for following a scalp.

5. The transducer according to claim 3, wherein the bottom of any or all of the one or more transducer modules are shaped as hexagons and/or pentagons.

6. The transducer according to claim 1, wherein the one or more transducer modules comprises:
a plurality of transducer modules configured to be positioned adjacent to each other on a scalp of a subject for TMS.

7. The transducer according to claim 6, wherein bottoms of the plurality of transducer modules form one or more patterns of a pentagon surrounded with one or more hexagons and/or one or more hexagons.

8. The transducer according to claim 6, wherein bottoms of the plurality of transducer modules form one or more patterns of a hexagon surrounded with three hexagons and three pentagons

9. The transducer according to claim 6, wherein bottoms of the plurality of transducer modules form one or more patterns of a central transducer module encircled by a plurality of encircling transducer modules, wherein the bottom for any or all of the plurality of encircling transducer modules has an irregular shape for extending a scalp footprint of a corresponding transducer module away from the central transducer module.

10. The transducer according to claim 6, configured for simultaneously activating any or all of the five or more coils of two or more neighboring transducer modules of the plurality of transducer modules for stimulating a single cortical target.

11. The transducer according to claim 1, wherein the five or more coils are configured for generating a signal space of five or more dimensions with each of the five or more coils corresponding to an orthogonal basis vector of the signal space.

12. The transducer according to claim 1, wherein the five or more coils for any or all of the one or more transducer modules have a coupling coefficient for mutual inductance less than 0.1.

13. The transducer according to claim 1, wherein the five or more coils comprises:
a round coil.

14. The transducer according to claim 1, wherein the five or more coils comprises:
two figure-of-eight coils perpendicular with respect to each other.

15. The transducer according to claim 1, wherein the five or more coils comprises:
two four-leaf clover coils rotated about a common longitudinal axis.

16. The transducer according to claim 1, wherein the five or more coils have same axis of symmetry.
